(19) United States
(12) Patent Application Publication
(10) Pub. No.: US 2026/0131077 A1
(43) Pub. Date: May 14, 2026

(54) SYSTEM FOR PERFORMING A CONTINUOUS INFUSION PROCESS USING AT LEAST TWO INFUSION DEVICES

(71) Applicant: Fresenius Vial SAS, Brézins (FR)

(72) Inventor: Rémy Wolff, Tullins (FR)

(21) Appl. No.: 19/122,821

(22) PCT Filed: Dec. 14, 2023
(86) PCT No.: PCT/EP2023/085748

§ 371 Date: Apr. 21, 2025

(30) Foreign Application Priority Data
Dec. 16, 2022 (EP) 22315329.7

(51) Int. Cl. A61M 5/168; A61M 5/142; A61M 5/145

(52) U.S. Cl. CPC A61M 5/16827; A61M 5/1452; A61M 2005/14208; A61M 2205/3334; A61M 2205/502

(57) ABSTRACT

A system (1) for performing a continuous infusion process using at least two infusion devices (10A, 10B) comprises: a first infusion device (10A) configured to adjustably deliver fluid (F1) from a first medication container (100A), a second infusion device (10B) configured to adjustably deliver fluid (F2) from a second medication container (100B), and a control system (11) configured to control the first and second infusion devices (10A, 10B) so that the first infusion device (10A) delivers fluid (F1) at a first time period and the second infusion device (10B) delivers fluid (F2) at a second time period. Therein, it is provided that the control system (11) comprises memory (110) storing a plurality of pre-defined transitional flow routines (R1-R5), and is configured to: select one of the plurality of pre-defined transitional flow routines (R1-R5) and control the first and second infusion devices (10A, 10B) in a transitional period (TP) between the first time period and the second time period in accordance with the selected pre-defined transitional flow routine (R1-R5).

The invention relates to a system for performing a continuous infusion process using at least two infusion devices.

A system with multiple infusion devices may be used to continuously administer a medication to a patient over a comparatively long period of time at a relatively constant dose rate. For this, however, it is necessary that the infusion devices are operated in a controlled, concerted fashion such that, upon depletion of a medication container associated with a first infusion device, infusion by a second infusion device from a second medication container starts in order to ensure a continuous, non-interrupted infusion process.

In this regard, WO 2017/012781 A1 provides a solution to a potentially problematic situation when a flow rate of the second infusion device is different than a flow rate of the first infusion device, leading to an increase or decrease in the dose rate by which residual medication in a common outlet delivery line is delivered to the patient.

EP 3 222 307 A1 describes start-up routines for one infusion device.

It is an ongoing task to optimize the transition of the delivery of the medication from the first infusion device to the second infusion device.

It is an object of the instant invention to provide a further improved system for performing a continuous infusion process using at least two infusion devices.

This object is achieved by means of a system comprising the features of claim 1.

Accordingly, a system for performing a continuous infusion process using at least two infusion devices is provided, the system comprising: a first infusion device configured to adjustably deliver fluid from a first medication container (e.g., to a common delivery line), a second infusion device configured to adjustably deliver fluid from a second medication container (e.g., to the common delivery line), and a control system configured to control the first and second infusion devices so that the first infusion device delivers fluid at a first time period and the second infusion device delivers fluid at a second time period. It is provided that the control system comprises memory storing a plurality of pre-defined transitional flow routines, and the system is configured to: select one of the plurality of pre-defined transitional flow routines and control the first and second infusion devices in a transitional period between the first time period and the second time period in accordance with the selected pre-defined transitional flow routine.

This is based on the following findings. Generally, many infusion devices such as syringe pumps are very accurate and can provide a very steady flowrate. Nevertheless, one disadvantage of such devices is commonly an uncertainty of their start-up flowrate. To start infusing, in the example of a syringe pump a pusher device pushes a piston of the syringe to move it at a desired speed. But as the syringe piston is commonly tightly inserted in the syringe barrel, some friction exists between a rubber part of the syringe piston, also called syringe stopper, and the syringe barrel. This friction is usually considered as a mix between static friction and dynamic friction. The static friction leads to a delay in the piston movement. As the kinematics chain between an electric drive device and the syringe pusher device cannot be infinitely rigid, the friction of the syringe will tense the elastic chain until the elastic force is equivalent to the static friction force that was retaining the syringe piston. At this moment, the syringe piston will move forward and, as the dynamic friction force is usually lower than the static one, one part of the elastic deformation of the power train will be cancelled when a part of this initial deformation will remain. As a consequence, the flowrate may first be delayed, and then partially compensated, giving a succession of underdose and overdose. In many cases this effect is small enough to be neglected. However, for certain drugs, such as nor-adrenaline, even a small overdose may be problematic. For other drugs, however, an underdose is more problematic than an overdose. This is addressed by the proposed system which allows a medication-dependent transition which enables an optimized transition for any drug.

The control system, in particular the (or a) memory of the control system may store a drug library associating each of a plurality of drugs with one or more of the plurality of pre-defined transitional flow routines and/or with a respective maximum flow rate. This allows to automatically present or select a suitable transitional flow routine for a given drug. Thereby, accidentally wrong selections of transitional flow routines can be avoided. In general, the control system can be adapted to receive an indication of a fluid type of the fluid (in particular, drug) in the first and/or second medication containers, and to select the one of the plurality of pre-defined transitional flow routines based on the indication of the fluid type.

The control system may be adapted to receive an indication of a drug and, optionally, to select the one of the plurality of pre-defined transitional flow routines based on the indication of the drug, in particular using the drug library. Thereby, selection of an appropriate transitional flow routine can be particularly simple and fail safe.

The system may comprise an interface to receive a user input, e.g., to receive the indication. Optionally, the control system is adapted to select the one of the plurality of pre-defined transitional flow routines based on the user input. This allows an easy and intuitive selection. The interface may comprise an output means, such as a display, and/or an input means, such as a button.

The control system may be adapted to disable a selection of at least one of the plurality of pre-defined transitional flow routines for at least one drug stored in the drug library. Thus, when a certain drug is used for infusion, inappropriate transitional flow routines can be disabled to avoid accidental selection thereof.

The first infusion device and/or the second infusion device may comprise an electric drive device. The electric drive device of the first infusion device and/or of the second infusion device may be configured to move a pusher device in a pushing direction. Alternatively, or in addition, first infusion device and/or the second infusion device may comprise one or more measuring device(s) for measuring one or more parameters associated with the pusher device. This allows to control a smooth transition from the first infusion device to the second infusion device.

The one or more parameters associated with the pusher device may comprise one, two, three or all of the following: a pushing distance travelled by the pusher device in the pushing direction, a force exerted by the pusher device in the pushing direction, a fluid volume delivered by movement of the pusher device in the pushing direction and a duration of movement of the pusher device in the pushing direction. For example, the parameters associated with the pusher device may comprise a pushing distance travelled by the pusher device in the pushing direction and a force exerted by the pusher device in the pushing direction. These parameters allow a precise control.

One of the pre-defined transitional flow routines may be an overdose-avoidance transitional flow routine. The overdose-avoidance transitional flow routine may be adapted to avoid exceeding a predefined flow rate. For example, according to the overdose-avoidance transitional flow routine, by means of the control system, in a first phase of the transitional period the electric drive device of the second infusion device is controlled to drive the pusher device to move at a first velocity, and, if the measured one or more parameters associated with the pusher device of the second infusion device exceeds a pre-defined threshold, the electric drive device of the second infusion device is controlled to drive the pusher device to move at a second velocity smaller than the first velocity. Thus, the pusher device of the second infusion device can be quickly brought into operative connection with the piston such that the delivery of the fluid from the medical container can start. A play in between the pusher device and the piston and an elasticity in the second infusion device are overcome such that an effective force transfer from the pusher device to the piston can be achieved. On the other hand, using the pre-defined threshold (which may be lower than a maximum value of the parameter, e.g., a force or distance) allows to avoid a (too high) peak flow rate. The first infusion device may be controlled to ramp down its pusher device velocity either progressive or instantaneously, at flow rates avoiding an overdose.

The one or more parameters of the pusher device may comprise a force exerted by (and/or a distance travelled by) the pusher device in the pushing direction. One of the pre-defined transitional flow routines may be an underdose-avoidance transitional flow routine. The underdose-avoidance transitional flow routine may be adapted to avoid decreasing below a predefined flow rate. For example, according to the underdose-avoidance transitional flow routine, by means of the control system, in a first phase of the transitional period the electric drive device of the second infusion device is controlled to drive the pusher device to move at a first velocity, and, optionally, if the measured parameter (e.g., force) exerted by the pusher device of the second infusion device is observed to correspond to (e.g., exceed or fall below) a predefined threshold, the electric drive device is controlled to drive the pusher device of the second infusion device to move at a second velocity, e.g., smaller than the first velocity. Again, the first infusion device may be controlled to ramp down its pusher device velocity either progressive or instantaneously, at flow rates avoiding an underdose.

The pre-defined threshold of the underdose-avoidance transitional flow routine may be larger than the pre-defined threshold of the overdose-avoidance transitional flow routine. This allows a simple but reliable configuration.

As an example, the first velocity may lie in the range between 0.05 mm/s to 2.5 mm/s, for example in between 0.1 mm/s to 0.15 mm/s. These velocities allow a safe operation.

Optionally, one of the pre-defined transitional flow routines is a smooth-overlap transitional flow routine. According to the smooth-overlap transitional flow routine, by means of the control system, the first infusion device may be controlled to gradually reduce a flow rate at which the fluid is delivered from the first medication container, and the second infusion device may be controlled to gradually increase a flow rate at which the fluid is delivered from the second medication container. This allows to both avoid too large overdoses and too large underdoses.

Optionally, one of the pre-defined transitional flow routines is an overdose-minimizing smooth-overlap transitional flow routine. According to the overdose-minimizing smooth-overlap transitional flow routine, by means of the control system, at a first instance of time the first infusion device is controlled to reduce a flow rate at which the fluid is delivered from the first medication container by a fraction of a target flow rate, and the second infusion device is controlled to start delivering fluid from the second medication container at a flow rate corresponding to the fraction of the target flow rate, and at a second instance of time the first infusion device is controlled to stop delivering fluid and the second infusion device is controlled to increase the flow rate to the target flow rate. This allows to administer fluid at an almost constant flow rate even during the transition. Alternatively, or in addition, one of the pre-defined transitional flow routines is a transitional flow routine according to which, by means of the control system, at a first instance of time the second infusion device is controlled to start delivering fluid from the second medication container and at a second instance of time after the first instance of time the first infusion device is controlled to reduce or stop delivering fluid from the first medication container. That is, according to at least one transitional flow routine, the second infusion device may be started before modifying the first infusion device flow rate.

For example, the fraction can be between 1/20 and ½. In particular, the fraction may be 1/10. This allows to overcome the static friction of the second infusion device with a little impact on the total fluid flow rate.

The first infusion device and/or the second infusion device may be constituted as a syringe pump (each). For syringe pumps, the advantages described herein are particularly prominent.

Further, the first medication container and/or the second medication container may be constituted by a cylindrical barrel of a syringe (each).

The control system may be adapted to set the length of the transitional period based on a flow rate of the first infusion device and/or the second infusion device.

The idea underlying the invention shall subsequently be described in more detail by referring to the embodiments shown in the figures. Herein:

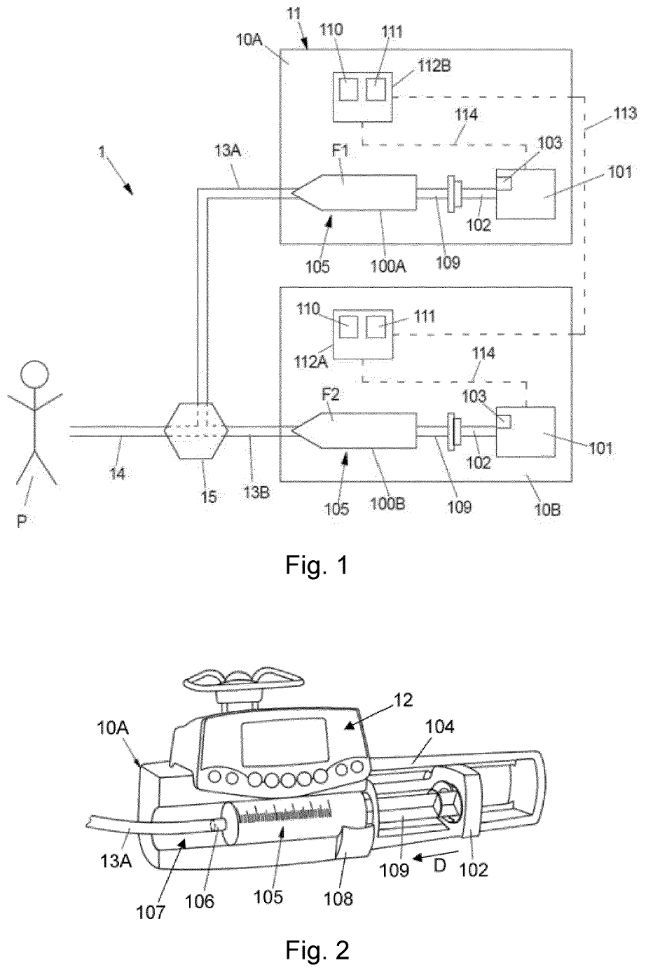

FIG. 1 shows a system for performing a continuous infusion process using at least two infusion devices;

FIG. 2 shows an infusion device of the system according to FIG. 1;

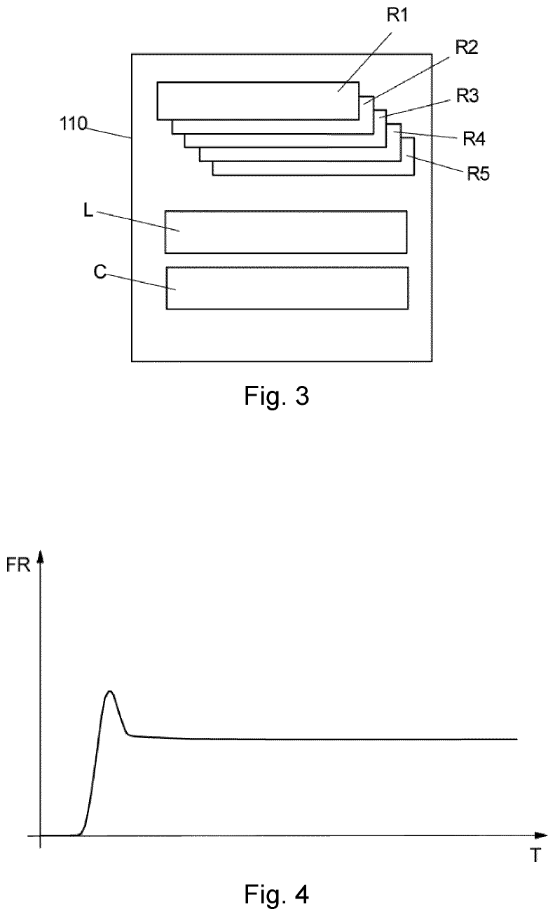

FIG. 3 shows a memory of the infusion device according to FIG. 2;

FIG. 4 shows a flow rate of a fluid flow of an infusion device of the system of FIG. 1;

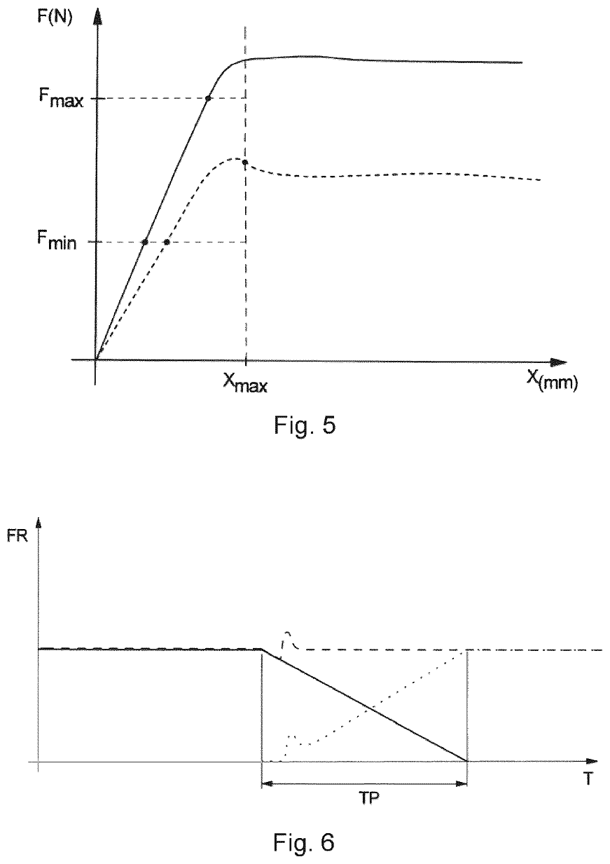

FIG. 5 shows a force applied by a pusher device of an infusion device of the system of FIG. 1;

FIG. 6 shows flow rates versus time using the system of FIG. 1 with a smooth-overlap transitional flow routine;

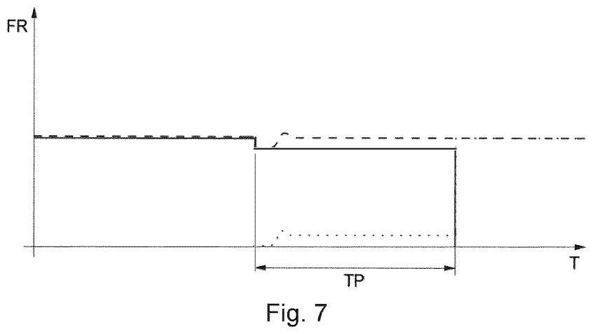

FIG. 7 shows flow rates versus time using the system of FIG. 1 with an overdose-minimizing smooth-overlap transitional flow routine; and

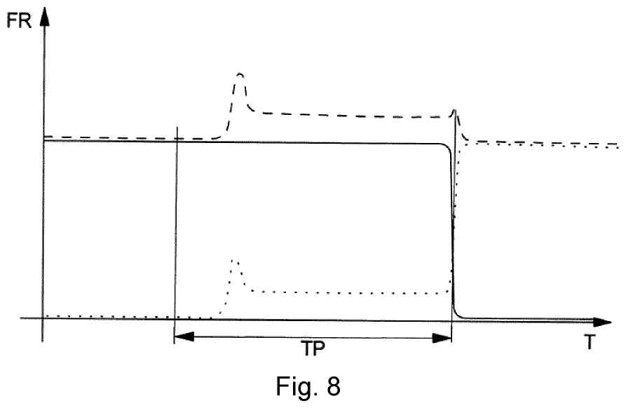

FIG. 8 shows flow rates versus time using the system of FIG. 1 with another transitional flow routine.

Subsequently, a system for performing a continuous infusion process using at least two infusion devices 10A, 10B shall be described. The embodiments described herein shall not be construed as limiting for the scope of the invention.

FIG. 1 shows a schematic drawing of a system 1 comprising, in the shown embodiment, two infusion devices 10A, 10B, namely a first infusion device 10A and a second infusion device 10B. The first infusion device 10A is configured to adjustably deliver fluid F1 from a first medication container 100A. The second infusion device 10B is configured to adjustably deliver fluid F2 from a second medication container 100B. The fluids F1, F2 in the first and second medication containers 100A, 100B may be of the same type (e.g., the same drug).

In the present example, the first infusion device 10A is configured to adjustably deliver the fluid F1 from the first medication container 100A via a first delivery line 13A to a common delivery line 14 towards a patient P, and the second infusion device 10A is configured to adjustably deliver the fluid F2 from the second medication container 100B via a second delivery line 13B to the common delivery line 14 towards the patient P.

Each of the infusion devices 10A, 10B is constituted as a syringe pump and comprises a syringe 105, which may be exchangeable. Each syringe 105 comprises a cylindrical barrel constituting the respective medication container 100A, 100B and a piston 109 received in the cylindrical barrel. The respective syringe 105 is received for example in a suitable holding device of the associated infusion device 10A, 10B. Each infusion device 10A, 10B comprises an electric drive device 101 for continuously pushing the respective piston 109 into the cylindrical barrel to deliver the respective fluid F1, F2 contained in the respective cylindrical barrel, e.g., at a constant flow rate towards the patient P.

The system 1 further comprises a control system 11 configured to control the first and second infusion devices 10A, 10B so that the first infusion device 10A delivers fluid F1 at a first time period and the second infusion device 10B delivers fluid F2 at a second time period. The control system 11 generally comprises memory 110 and at least one processor 111. In the example shown, the control system 11 comprises a first control device 112A being a part of the first infusion device 10A and a second control device 112B being a part of the second infusion device 10B. Each of the first and second control devices 112A, 112B comprises memory 110 and a processor 111. The first and second control devices 112A, 112B are communicatively coupled via a communication line 113. However, in other examples, the control system 11 may be located at only one of the control devices 112A, 112B, or may be formed as or in a device external to both the first and second infusion devices 10A, 10B. The control system 11 is communicatively coupled with the electric drive devices 101 of the first and second infusion devices 10A, 10B via respective communication lines 114. Thus, the electric drive devices 101 of the first and second infusion devices 10A, 10B are controlled by the control system 11.

The system 1 is set up to perform a continuous infusion operation. For this, the first and second delivery lines 13A, 13B both lead to the common delivery line 14. This connection may be a direct fluid connection, e.g., with a Y-shaped adapter. An optional relay device 15 may control flow from the first and second delivery lines 13A, 13B to the common delivery line 14. The common delivery line 14 may, for example, be connected to the patient P by means of a suitable injection needle or the like such that via the common delivery line 14 the fluid F1, F2 from the first and second medication containers 100A, 100B can be administered to the patient P.

The (continuous) infusion process starts, for example, in a first infusion phase with the first infusion device 10A delivering fluid F1, in particular, a medication, from its first medication container 100A via the first delivery line 13A at a first flow rate such that the fluid F1 is delivered to the patient P at the first flow rate.

For example, once the first medication container 100A is nearly depleted, the infusion process shall be switched over to the second infusion device 10B.

At the end of the first infusion phase (e.g., when the first medication container 100A is nearly depleted) a control signal is issued by for example the processor 111 of the first infusion device 10A and communicated to the processor 111 of the second infusion device 10B, the control signal causing the operation of the first infusion device 10A to stop and the operation of the second infusion device 10B to start. Optionally, at the same time, also the relay device 15 is switched (for example by switching a suitable flow switching means contained in the relay device 15).

To optimize the transition for the various possible drugs for infusion, the control system 11 comprises memory 110 (e.g., the memory 110 of the first infusion device 10A and/or the memory of the second infusion device 10B and/or a memory external thereof) storing a plurality of pre-defined transitional flow routines R1-R5, and is configured to: select one of the plurality of pre-defined transitional flow routines R1-R5 and control the first and second infusion devices 10A, 10B in a transitional period TP between the first time period and the second time period in accordance with the selected pre-defined transitional flow routine R1-R5.

Each of the infusion devices 10A, 10B comprises at least one measuring device 103 for measuring one or more parameters, e.g. a pushing distance X (see FIG. 5) travelled by the pusher device 102 in a pushing direction D, a force F (see FIG. 5) exerted by the pusher device 102 in the pushing direction D, a fluid volume delivered by movement of the pusher device 102 in the pushing direction D and/or a duration of movement of the pusher device 102 in the pushing direction D. Using the one or more parameters, the transition between the first infusion device and the second infusion device may be improved.

While FIG. 1 shows the infusion devices 10A, 10B very schematically, FIG. 2 illustrates an optional more specific design of the first infusion device 10A. The first and second infusion devices 10A, 10B may have the same design. The first infusion device 10A has a housing 104 and a receptacle 107 arranged on the housing 104 to receive the syringe 105 therein. The cylindrical barrel of the syringe 105 is connected, via a connector 106, to the first delivery line 13A.

For installing the syringe 105 on the receptacle 107 of the first infusion device 10A, the cylindrical barrel of the syringe 105 is placed in the receptacle 107 and is mechanically connected to the housing 104 by means of a fixation device 108. By means of the fixation device 108, for example constituted by a releasable clamp element, the cylindrical barrel is secured within the receptacle 107 such that the cylindrical barrel is held in position on the receptacle 107.

For delivering medical fluid F1 contained in the cylindrical barrel, the piston 109 of the syringe 105 can be pushed into the cylindrical barrel in a pushing direction D. For this, the first infusion device 10A comprises a pusher device 102 movably arranged within a guide device and connected to the electric drive device 101.

For operating the first infusion device 10A, the syringe 105 is installed and the pusher device 102 is (manually) moved towards a piston head of the piston 109 until the pusher device 102 comes into abutment with the piston head. For performing an infusion process the pusher device 102 is then electrically moved in the pushing direction D to move the piston 109 into the cylindrical barrel for delivering the fluid F1 contained in the cylindrical barrel via the delivery line 13A towards the patient P.

The first infusion device 10A further comprises a human-machine interface 12 for outputting information and inputting commands.

FIG. 3 shows contents of the memory 110 of the control system 11, e.g., the memory 110 of the first and/or second infusion device 10A, 10B.

The memory 110 stores plurality of pre-defined transitional flow routines R1-R5 and a drug library L, which will be described in further detail below.

The memory 110 further stores instructions C that, when executed by one or more processors of the control system 11, e.g., the processors 111 of the first and second infusion devices 10A, 10B, cause the processors to select one of the stored plurality of pre-defined transitional flow routines R1-R5 and to control the first and second infusion devices 10A, 10B in a transitional period TP between the first time period and the second time period in accordance with the selected pre-defined transitional flow routine R1-R5.

FIG. 4 is a diagram of a flow rate FR of one of the infusion devices 10A, 10B of FIG. 1 versus time T and illustrates the effect of static and dynamic friction in syringe pumps. As mentioned further above, the syringe piston 109 is commonly tightly inserted in the syringe barrel, the friction between these parts is a mix of static friction and dynamic friction. The static friction leads to a delay in the piston movement as can be seen in the left part of the diagram. As soon as the elastic force is equivalent to the static friction force that was retaining the syringe piston, the syringe piston 109 moves forward more quickly, so the flow rate FR is first delayed, and then partially compensated, giving a succession of underdose and overdose, the latter being visible as a peak in FIG. 4. After this start-up phase, the flow rate FR is relatively constant.

Due to this succession of underdose and overdose, it can be a challenge to make the transition as smooth as possible. In addition, some drugs may not be administered with an overdose, others may not be administered with an underdose. Further drugs may tolerate small overdoses or small overdoses but may require in general a flow rate being as constant as possible.

For example, for some drugs with a long half-life, the precise instant when the drug is administrated is not predominantly important. For those drugs, what commonly matters is mainly the quantity and the rate. If the administration starts 5 minutes sooner or later than initially planned, this may have little or no effect on the efficiency of the therapy. For some other drugs, their effect is so quick and sometimes so critical that it is important to make sure that the patient receives the drug immediately when it is decided by the healthcare team. It could be preferable, for such drug, to receive more but in right time than to receive the correct dose but with a delay. For some other drugs, on the opposite, a toxic effect could be feared in case the infused dose exceeded the expected dose at start. These issues are also present in the transition in a channel relay.

To address these issues, the memory 110 stores a plurality of pre-defined transitional flow routines R1-R5. In this example, the plurality of pre-defined transitional flow routines R1-R5 comprises:
an overdose-avoidance transitional flow routine R1,
an underdose-avoidance transitional flow routine R2,
a smooth-overlap transitional flow routine R3,
an overdose-minimizing smooth-overlap transitional flow routine R4 and
another transitional flow routine R5.

Turning now to FIG. 5, the overdose-avoidance transitional flow routine R1 and the underdose-avoidance transitional flow routine R2 will be described. Two curves illustrate two independent example pumping processes of the second infusion device 10B.

FIG. 5 shows the force F exerted by the pusher device 102 of the second infusion device 10B in the pushing direction D versus the pushing distance X travelled by the pusher device 102 in the pushing direction D.

With increasing pushing distance X, first, the resulting force F increases until a maximum force is reached (when the static friction force is reached). Thereafter, the force F eventually decreases (see the lower example curve) and then remains constant.

To avoid an overdose, the overdose-avoidance transitional flow routine R1 is conducted as follows (see the lower curve of FIG. 5): in a first phase of the transitional period TP the electric drive device 101 of the second infusion device 10B is controlled to drive the pusher device 102 to move at a first velocity to more quickly pass the underdose region, and, if (a) measured parameter(s) associated with the pusher device 102 of the second infusion device 10B, in this example the force F, exceeds a pre-defined threshold F_{MIN}, the electric drive device 101 of the second infusion device 10B is controlled to drive the pusher device 102 to move at a second velocity. The first velocity is larger than the second velocity. At the same time, the first infusion device may correspondingly ramp down its flow rate.

Optionally, conducting the overdose-avoidance transitional flow routine R1 (or the underdose-avoidance transitional flow routine R2 or yet another transitional flow routine) comprises monitoring a distance X travelled by the pusher device 102 of the second infusion device 10B. The overdose-avoidance (or other) transitional flow routine R1 may comprise comparing the distance X travelled with a pre-defined threshold distance X_{MAX}. Exceeding the pre-defined threshold distance X_{MAX} may trigger a change of the velocity of the pusher device 102. For example, when the threshold F_{MIN} force has not yet been reached but the threshold distance X_{MAX} is already reached, the electric drive device 101 may be controlled to drive the pusher device 102 of the second infusion device 10B to move at the second velocity.

To avoid an underdose, on the other hand, the underdose-avoidance transitional flow routine R2 is conducted as follows (see the upper curve of FIG. 5): in a first phase of the transitional period TP the electric drive device 101 of the second infusion device 10B is controlled to drive the pusher device 102 to move at a (large) first velocity (e.g., the same as in the overdose-avoidance transitional flow routine R1), and, if the measured force F exerted by the pusher device 102 of the second infusion device 10B is observed to exceed a pre-defined threshold FMAX, the electric drive device 101 is controlled to drive the pusher device 102 of the second infusion device 10B to move at a (low) second velocity (e.g., the same as in the overdose-avoidance transitional flow routine R1). At the same time, the first infusion device may correspondingly ramp down its flow rate.

The threshold F_{MAX} of the underdose-avoidance transitional flow routine R2 is larger than the threshold F_{MIN} of the overdose-avoidance transitional flow routine R1.

The first velocity may be in the range between 0.05 mm/s to 2.5 mm/s, for example in between 0.1 mm/s to 0.15 mm/s.

Turning now to FIG. 6, the smooth-overlap transitional flow routine R3 will be described. FIG. 6 shows the flow rate FR of the first infusion device 10A (solid line), the flow rate FR of the second infusion device 10B (dotted line), and the total flow rate FR on the first and second infusion devices 10A, 10B (dashed line) versus time T.

The smooth-overlap transitional flow routine R3 is conducted as follows: the first infusion device 10A is controlled to gradually (here: linearly) reduce a flow rate FR at which the fluid F1 is delivered from the first medication container 100A over the transitional period TP, and the second infusion device 10B is controlled to gradually (here: neglecting the start-up phase, linearly) increase a flow rate FR at which the fluid F2 is delivered from the second medication container 100B. The result is a relatively constant flow rate FR before, during and after the transitional period TP. However, small overdoses can remain. This can be neglected for some drugs, but for others not.

Thus, FIG. 7 illustrates the overdose-minimizing smooth-overlap transitional flow routine R4, according to which: at a first instance of time the first infusion device 10A is controlled to reduce a flow rate FR at which the fluid F1 is delivered from the first medication container 100A by a fraction (e.g., 10% or 5%) of a target flow rate FR, and the second infusion device 10B is controlled to start delivering fluid F2 from the second medication container 100B at a flow rate FR corresponding to the fraction of the target flow rate. Thus, the first infusion device 10A is stepped down the e.g. 90% and the second infusion device 10B is stepped up to 10%, so the sum remains at 100%. At a later, second instance of time the first infusion device 10A is controlled to stop delivering fluid F1 and the second infusion device 10B is controlled to increase the flow rate FR to the target flow rate (100%). By this, the effect of the start-up can be strongly diminished, and an almost constant total flow rate FR is possible. By this, it can be made sure that the total flow rate FR does not fall below a given fraction, e.g. 90%.

Optionally, the length of the transitional period TP is dependent on the (target) flow rate FR. The transitional period is long enough to make sure that the second infusion device 10B is ramped up before the first infusion device 10A is stopped. For example, the transitional period TP may last 5 minutes or less, or 10 minutes or less. Ramping up and/or down may be controlled in steps (e.g., 10 steps of 30 s)

According to the further transitional flow routine R5, at a first instance of time the second infusion device 10B is controlled to start delivering fluid F2 from the second medication container 100B and at a second instance of time after the first instance of time the first infusion device 10A is controlled to reduce or stop delivering fluid F1 from the first medication container 100A. This may be combined with any of the other transitional flow routines R1-R4. For example, by this the small underdose shown in FIGS. 6 and 7 can be reduced or avoided.

Such a flow routine is shown in FIG. 8. Therein, pumping of the first infusion device 10A is unchanged while at the first instance of time the second infusion device 10B is controlled to start delivering fluid F2 from the second medication container 100B (see dotted line). Thus, in the transitional period TP starting at the first instance of time, the total flow rate FR (see dashed line) does not fall below a target flow rate, so an underdose is avoided. To minimize an overdose, however, the second infusion device 10B is controlled to start delivering fluid from the second medication container 100B at a flow rate corresponding to a fraction of the target flow rate.

At the second instance of time simultaneously the first infusion device 10A is controlled to stop (or ramp down) delivering fluid F1 from the first medication container 100A (see solid line) and the second infusion device 10B is controlled to start (or ramp up) delivering fluid F2 from the second medication container 100B (see dotted line) at the target flow rate. The delay between the first instance of time and the second instance of time is pre-defined. The delay is set such that the flow rate FR peak of the second infusion device 10B occurs within the transitional period TP. This flow routine may also be referred to as a underdose-avoiding smooth-overlap transitional flow routine.

Some or all of these transitional flow routines R1-R5 may be stored in the memory 110.

Optionally, a transitional flow routine R1-R5 may be selected by a command entered into the interface 12. Alternatively, or in addition, the system 1 may comprise the drug library L. The drug library L associates each of a plurality of drugs with a respective one of the plurality of pre-defined transitional flow routines R1-R5. Thus, when the drug in the medication containers 100A, 100B is entered via the interface 12 or automatically detected by the first and/or second infusion devices 10A, 10B, the correct transitional flow routine R1-R5 can be selected. On the other hand, the control system 11 can be adapted to disable selection of at least one of the plurality of pre-defined transitional flow routines R1-R5 for at least one drug stored in the drug library L. Thus, transitional flow routines R1-R5 that are not suitable for a given drug may not be accidentally selected.

The system 1 may also have a drug error reduction system, wherein for a given drug, a respective maximum flow rate FR may be stored in the drug library L.

Hence, the present solutions allows a caregiver to manually or automatically select an appropriate routine based on the drug to be administered.

The idea of the invention is not limited to the embodiments described above but may be implemented in a different fashion.

### LIST OF REFERENCE NUMERALS

1 System
10A, 10B Infusion device
100A, 100B Medication container
101 Electric drive device
102 Pusher device
103 Measuring device
104 Housing
105 Syringe
106 Connector
107 Receptacle
108 Fixation device
109 Piston
11 Control system
110 Memory
111 Processor
112A, 112B Control device
113 Communication line
114 Communication line
12 Interface
13A, 13B Delivery line
14 Common delivery line
15 Relay device
C Instructions
D Pushing direction
F Force
F1, F2 Fluid
F_{MIN}, F_{MAX} Threshold
FR Flow rate
L Drug library
P Patient
R1-R5 Transitional flow routine
T Time
TP Transitional period
X, X_{MAX} Distance

1. A system for performing a continuous infusion process using at least two infusion devices, the system comprising:
a first infusion device configured to adjustably deliver fluid from a first medication container,
a second infusion device configured to adjustably deliver fluid from a second medication container, and
a control system configured to control the first and second infusion devices so that the first infusion device delivers fluid at a first time period and the second infusion device delivers fluid at a second time period,
wherein
the control system comprises memory storing a plurality of pre-defined transitional flow routines, and is configured to:
one of the plurality of pre-defined transitional flow routines and
control the first and second infusion devices in a transitional period between the first time period and the second time period in accordance with the selected pre-defined transitional flow routine.

2. The system according to claim 1, wherein the memory of the control system stores a drug library associating each of a plurality of drugs with one or more of the plurality of pre-defined transitional flow routines and/or with a respective maximum flow rate.

3. The system according to claim 2, wherein the control system is configured to receive an indication of a drug and to select the one of the plurality of pre-defined transitional flow routines based on the indication of the drug using the drug library.

4. The system according to claim 1, further comprising an interface to receive a user input, wherein the control system is configured to select the one of the plurality of pre-defined transitional flow routines based on the user input.

5. The system according to claim 2, claim further comprising an interface to receive a user input, wherein the control system is configured to select the one of the plurality of pre-defined transitional flow routines based on the user input, and wherein the control system is configured to disable selection of at least one of the plurality of pre-defined transitional flow routines for at least one drug stored in the drug library.

6. The system according to claim 1, wherein the first infusion device and/or the second infusion device comprise an electric drive device configured to move a pusher device in a pushing direction and at least one measuring device for measuring one or more parameters associated with the pusher device.

7. The system according to claim 6, wherein the one or more parameters associated with the pusher device comprise one or more of:
a pushing distance travelled by the pusher device in the pushing direction,
a force exerted by the pusher device in the pushing direction,
a fluid volume delivered by movement of the pusher device in the pushing direction, and
a duration of movement of the pusher device in the pushing direction.

8. The system according to claim 6, characterized wherein one of the pre-defined transitional flow routines is an overdose-avoidance transitional flow routine, according to which, by means of the control system:
in a first phase of the transitional period the electric drive device of the second infusion device is controlled to drive the pusher device to move at a first velocity, and
if the measured one or more parameters associated with the pusher device of the second infusion device exceeds a pre-defined threshold, the electric drive device of the second infusion device is controlled to drive the pusher device to move at a second velocity smaller than the first velocity.

9. The system according to one of claim 6, wherein the one or more parameters associated with the pusher device comprise a force exerted by the pusher device in the pushing direction, wherein one of the pre-defined transitional flow routines is an underdose-avoidance transitional flow routine, according to which, by means of the control system:
in a first phase of the transitional period the electric drive device of the second infusion device is controlled to drive the pusher device to move at a first velocity, and
if the measured force exerted by the pusher device of the second infusion device is observed to correspond to a pre-defined threshold, the electric drive device is controlled to drive the pusher device of the second infusion device to move at a second velocity smaller than the first velocity.

10. The system according to claim 8, wherein the one or more parameters associated with the pusher device comprise a force exerted by the pusher device in the pushing direction, wherein one of the pre-defined transitional flow routines is an underdose-avoidance transitional flow routine, according to which, by means of the control system:
in a first phase of the transitional period the electric drive device of the second infusion device is controlled to drive the pusher device to move at a first velocity, and
if the measured force exerted by the pusher device of the second infusion device is observed to correspond to a pre-defined threshold, the electric drive device is controlled to drive the pusher device of the second infusion device to move at a second velocity smaller than the first velocity, and
the pre-defined threshold of the underdose-avoidance transitional flow routine is larger than the pre-defined threshold of the overdose-avoidance transitional flow routine.

11. The system according to claim 9, wherein the first velocity lies in the range between 0.05 mm/s to 2.5 mm/s.

12. The system according to claim 1, wherein one of the pre-defined transitional flow routines is a smooth-overlap transitional flow routine, according to which, by means of the control system:
the first infusion device is controlled to gradually reduce a flow rate at which the fluid is delivered from the first medication container, and
the second infusion device is controlled to gradually increase a flow rate at which the fluid is delivered from the second medication container.

13. The system according to claim 1, wherein one of the pre-defined transitional flow routines is an overdose-minimizing smooth-overlap transitional flow routine, according to which, by means of the control system
at a first instance of time the first infusion device is controlled to reduce a flow rate at which the fluid is delivered from the first medication container by a fraction of a target flow rate, and the second infusion device is controlled to start delivering fluid from the second medication container at a flow rate corresponding to the fraction of the target flow rate, and
at a second instance of time the first infusion device is controlled to stop delivering fluid and the second infusion device is controlled to increase the flow rate to the target flow rate;
and/or that one of the pre-defined transitional flow routines is a transitional flow routine according to which, by means of the control system:
at a first instance of time the second infusion device is controlled to start delivering fluid from the second medication container and
at a second instance of time after the first instance of time the first infusion device is controlled to reduce or stop delivering fluid from the first medication container.

14. The system according to claim 13, wherein the fraction is between 1/20 and ½.

15. The system according to claim 1, wherein the first infusion device and the second infusion device are each a syringe pump.

16. The system according to claim 11, wherein the first velocity lies in the range between 0.1 mm/s to 0.15 mm/s.

17. The system according to claim 14, wherein the fraction is 1/10.
